(19) United States
(12) Patent Application Publication
(10) Pub. No.: US 2007/0232911 A1
(43) Pub. Date: Oct. 4, 2007

(54) DEVICE FOR PHOTODETECTING TUMOR

(75) Inventor: Taeko Urano, Kawasaki-shi (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: KABUSHIKI KAISHA TOSHIBA, Tokyo (JP)

(21) Appl. No.: 11/686,744

(22) Filed: Mar. 15, 2007

(30) Foreign Application Priority Data
Mar. 30, 2006 (JP) 2006-095782

(51) Int. Cl. A61B 8/00

(52) U.S. Cl. 600/438; 600/476

(57) ABSTRACT

The present invention includes i) an irradiator which irradiates 1st light with wavelength within a photo-absorption band of glucose on organism tissues, ii) an detector which detects reflected light of the 1st light from the organism tissues, iii) an operation unit configured to operate distribution of level of glucose, and iv) a determination unit configured to determine presence of a tumor by analyzing the distribution of level of glucose.

### CROSS-REFERENCE TO RELATED APPLICATIONS

This application is based upon and claims the benefit of priority from prior Japanese Patent Application No. 2006-095782, filed Mar. 30, 2006, the entire contents of which are incorporated herein by reference.

### BACKGROUND OF THE INVENTION

1. Field of the Invention

The present invention relates to a tumor photo-detection device that can noninvasively examine an internal of a living body.

2. Description of the Related Art

Early detection of a tumor is very important for the treatment of the tumor, and therefore in order to detect a tumor in an early stage, various methods and technologies are employed.

First, a topologic diagnostic method is conventionally known (as discussed in, for example, JP-A 2005-328507 (KOKAI)). The topologic diagnostic method is a diagnostic method for detecting the position of a tumor based on a value obtained by measuring a physical constant (X-ray absorbance or acoustic impedance). More specifically, the X-ray absorbance and/or acoustic impedance of the organism tissues at various positions are measured, and an area that is different from the normal site within the living body is detected based on the difference in measured value. Then, the distribution state of neovasculars generated in the region and its peripheral region, and the size and shape of micro-calcification generated in the area are analyzed and thus the area is diagnosed whether it is a tumor or not. Such a topologic diagnostic method entails a drawback of, for example, being exposed to X-ray.

Next, a physiological diagnostic method is conventionally known (as discussed in, for example, JP-A 2005-164609 (KOKAI)). The physiological diagnostic method is a method of diagnosing a cancer cell by injecting into a living body glucose marked with an isotope which emits positron, and imaging the distribution of the injected glucose in the body with a special camera. As a specific example of this method, there is a PET (positron emission tomography) examination which can detect a cancer cell in an early stage. In the PET examination, the difference in physiological phenomenon between a cancer cell and a normal cell (that is, the phenomenon in which more glucose is taken into a cancer cell than into a normal cell) is focused. More specifically, with use of glucose marked with an isotope, a cancer cell which takes in more glucose, thereby emitting stronger radiation than that of the normal cell is specified. Such a physiological diagnostic method entails the problem of safety of the normal cell tissues since a radioactive substance is injected to the living body.

In recent years, in addition to the above-described topologic diagnostic method and physiological diagnostic method, an optical diagnostic method has become focus of attention (for example, as is discussed in JP-A-2004-5311311 (KOHYO)). The optical diagnostic method is a method of diagnosing the presence or absence of a tumor in a tissue by optically measuring the concentration of a specific biosubstance (tumor marker). The optical diagnostic method is free from such problems as the exposure to x-rays and the intake of radioactive substances, and it is superior to the other conventional methods in the aspect that the inside of a living body can be examined noninvasively.

The optical diagnostic method requires age correction. The age correction is to vary the standard value (normal value) of the concentration of a specific biosubstance (tumor marker) in accordance with the age of the patient. The concept of the age correction is based on the physiological phenomenon that the contents of the biosubstance is greatly varied along with age. The concentration of many biosubstances is influenced by aging, and therefore when one of these biosubstances is selected as the tumor marker, the age correction is essential. At present, all the tumor markers employed in the optical diagnostic method are substances that require the age correction.

### BRIEF SUMMARY OF THE INVENTION

The tumor photo-detection device according to the present invention includes the following structural elements:

i) an irradiator which irradiates first light with a wavelength within a photo-absorbing band of glucose and second light with a wavelength within a photo-absorbing band of water, on a plurality of regions of organism tissue;

ii) a detector which detects reflected light of the first light from the organism tissue and reflected light of the second light from the organism tissue;

iii) an operation unit configured to operate distributions of concentration levels of glucose and water in the plurality of regions by comparing the reflected light of the first light with that of the second light with regard to an increase or decrease in intensity; and

iv) a determination unit configured to determine presence or absence of an area where regions of high concentration level of glucose and regions of low concentration level of water appear alternately by analyzing the distributions of the concentration levels.

Additional objects and advantages of the invention will be set forth in the description which follows, and in part will be obvious from the description, or may be learned by practice of the invention. The objects and advantages of the invention may be realized and obtained by means of the instruments and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a tumor photo-detection device according to one embodiment;

FIG. 2 is a schematic diagram showing a probe of the tumor photo-detection device according to the embodiment;

FIG. 3A is a schematic diagram showing arrangement of a light-radiating fiber and an acoustic element of the tumor photo-detection device according to the embodiment;

FIG. 3B is a front view of the acoustic element 50 shown in FIG. 3A;

FIG. 4 is a schematic diagram showing the probe of the tumor photo-detection device according to the embodiment, rotating above a model sample; and

FIG. 5 is a top view of a model sample of a organism tissue.

### DETAILED DESCRIPTION OF THE INVENTION

The inventors of the present invention carried out intensive researches and studies on the optical diagnostic method in order to further improve the accuracy of the result of diagnosis. In the studies, a conclusion was drawn that the age correction is an obstacle to the further improvement of the accuracy of the diagnostic results for the following reason. That is, we cannot lead to the accuracy of the diagnostic results only using the real age of a patient and correcting the shift in the concentration of each biosubustance, because there is individual difference for the shift in the concentration by age. Further, the inventors of the present invention further discovered that the variation in the concentration of each biosubstance in the breast of a female differs even more greatly among individuals as compared to those of the other parts, and therefore the effect of the age correction cannot be expected very much. Based on the findings, the inventors carried out further researches and studies to develop an optical diagnostic method which does not require age correction.

That is, an object of the present invention is to provide a tumor photo-detection device that can examine an inside of a living body noninvasively without physical damages to the living body and can detect a tumor accurately without being influenced by the difference among individuals in aging.

As an achievement of the above-described object, the inventors have completed a truly novel tumor photo-detection device.

The tumor photo-detection device of the present invention comprises the following structural elements:

i) an irradiator which irradiates first light having a wavelength within a photo-absorbing band of glucose and second light having a wavelength within a photo-absorbing band of water, on a plurality of regions of organism tissue;

ii) a detector which detects reflected light of the first light from the organism tissue and reflected light of the second light from the organism tissue;

iii) an operation unit configured to operate concentration distributions of glucose and water in the plurality of regions by comparing the reflected light of the first light with that of the second light with regard to an increase or decrease in intensity; and

iv) a determination unit configured to determine presence or absence of a region where a high concentration of glucose and a low concentration of water appear alternately by analyzing the distributions of the concentration levels.

Embodiments of the present invention will now be described in detail with reference to accompanying drawings. Throughout the figures, the same structural part is designated by the same reference number and the explanation for the part is not repeated. Further, the figures are all schematically drawn and therefore the relation between the thickness and width, the proportions between layers in thickness, etc. may differ from an actual structure. Furthermore, among the figures, the measurements, proportions, etc. may differ in some parts.

First, compounds that are used as tumor markers will be explained.

In order for a compound to serve as a tumor marker, it is necessary that the compound should be of a type that exhibits different concentrations between a normal site and tumor site in organism tissue. Further, the compound should be of a type whose concentration within the organism tissue is not easily influenced by aging. With use of such a compound, it is no longer necessary to carry out the age correction. Therefore, it becomes possible to obtain with the tumor marker accurate data that support the presence of a tumor without having to consider aging, which is a factor that greatly differs among individuals. The “compound that is not easily influenced by aging” is a compound whose concentration differs between a normal site and tumor site within organism tissue and which is not easily influenced along with age.

A specific example of the compound is glucose. As described above, the concentration of glucose within a tumor cell is always higher than that of a normal cell, and this tendency does not vary along with age. Therefore, the concentration of glucose in organism tissue is optically measured, and thus a region where the glucose concentration is high is specified. In this manner, a tumor site can be specified without carrying out the age correction. Here, the glucose concentration obtained by the optical measurement can be utilized directly as data that support the presence of a tumor without carrying out the age correction. Here, in order to use glucose in the examination, it is necessary to use light with a wavelength within a photo-absorption band unique to glucose. More specifically, light with a wavelength in a photo-absorption range of 905 nm, 1450 nm, 1550 nm, 1640 nm or 2130 nm can be used.

It should be noted here that the glucose concentration in a tumor varies with actions of everyday life such as eating and exercising, as same as the glucose concentration in normal tissue. Therefore, for example, when the measurements are taken for both cases that water containing glucose is given or not given when fasting, the accuracy of the examination results can be further more improved.

Second, the principle of the measurement will now be described.

There are several means to measure the concentration of a specific compound present in a target region. Here, the intensity of absorbed light with a wavelength within the photo-absorption band unique to the compound is measured and thus the adverse effect of noise created by some other compound can be excluded. The intensity of absorbed light in the organism tissue can be calculated by measuring the intensity of reflected light with the wavelength within the photo-absorption band from the inside of the organism tissue. For such an operation unit, the structure of the measuring apparatus can be relatively simple. On the other hand, the optical energy accumulated on the compound by the photo-absorption generates heat and acoustic wave. The technique of measuring a generated acoustic wave using an acoustic element is called an optoacoustic method, which can effectively utilize the dynamic range as compared to the method of measuring the intensity of reflected light. Some other techniques are the detection of generated heat as infrared ray and the detection of a change in temperature as a change in sonic speed of ultrasonic wave.

In order to locate the position where a target compound (absorber) is present, the measurement is carried out by setting a plurality of positions from where the light (heat or acoustic wave) is emitted to the outside of the body, based on the position where the light is input to the body. When the light used in the measurement is continuous in time (that is, CW light), the space resolved method is employed. With this method, the position of the target compound is located by calculating the inverse problem with estimating optical path statistically. In the case where the light used in the measurement varies along with time (that is, modulated intensity of light, pulse light), the delay of the phase or arrival time of the measured light with respect to the input light corresponds to the length of the optical path (optical path length). Therefore, when the inverse problem is calculated based on the data obtained, the position of the absorber can be located. For the details of the structure of the apparatus, see reference document, for example, “Visualizing Technique of Bio-datall, Corona 1997, Phys. Med. Biol. Vol. 50, R1 to 43, 2005.

In the case where acoustic wave is to be measured, first, the location where an acoustic wave was generated is estimated. Then, the measurement of the acoustic wave is repeated at various measurement points, and the position of the absorber is determined at a location where there is no contradiction as a whole (see Appl. Opt. 39, 5872-83 (2000)). The amount of the absorber is assigned to the location of the absorber thus determined, and thus a spatial distribution diagram is formed.

The compound distributed in a tumor site is detected by utilizing its photo-absorption property as described above and the concentration and distribution shape are compared quantitatively with the threshold value. In this manner, it becomes possible not only to distinguish a tumor from a normal tissue but also to distinguish a malignant tumor from a benign tumor. Further, the optical measurement does not entail such a drawback of being exposed to and influenced by radioactivity. Therefore, in combination with other conventional treatment apparatus, the accuracy of the diagnosis can be significantly improved without physical loads to the patient.

Third, a tumor photo-detection device specialized for determining the presence/absence of breast cancer will be described.

The inventors made special effort on breast cancer among many types of cancers. The micro-calcification phenomenon that occurs during the process of development of breast cancer is utilized in the detection of the cancer. The micro-calcification phenomenon is a phenomenon in which calcium oxalate or calcium phosphate concentrates in the vicinity of mammary duct and creates an enormous number of aggregates. When micro-calcification areas gather in dots, calcification areas creates a spicular shape, or calcification areas are distributed along mammary duct, there are high possibilities of breast cancer. Therefore, when the above-described phenomenon is observed, it is necessary to re-measure at a higher special resolution (see Diagnosis and Treatment of Mammary Cancer—Latest Research Trends—Japan Clinical (2000)). The mammography has such a high spatial resolution as 100 μm or less, and it is capable of detecting a micro-calcification area as described above or its shape. The measurement based on light entails such a drawback that the spatial resolution is deteriorated due to multiple scatterings, and therefore it is difficult to measure a micro-calcification area itself to be focused. Since micro-calcification is a phenomenon that occurs in a part of tissue, it is possible to detect a micro-calcification phenomenon indirectly from its distribution tendency by measuring the compound distribution in its vicinity including the micro-calcification area. A micro-calcification area is a tumor region in which the activity of tumor cells is enhanced, whereas the metabolic activity of normal cells is suppressed. The composition of a micro-calcification tumor region is different from the composition of a non-calcification tumor region. For example, micro-calcification areas are characterized by their low water contents. In many cases, a large calcification area indicates that it is not malignant, whereas in a small calcification area in which a malignant tumor is suspected, areas with low water contents are non-continuously present in dots. Further, in the case where the non-continuous areas are distributed along mammary duct, these areas are invaded at high probability by carcinoma. Based on the above-described characteristics of the breast cancer, the tumor photo-detection device of the present invention can easily evaluate the presence or absence of occurrence of breast cancer, by measuring the photo-absorption distribution of glucose and water respectively, and determining the presence or absence of an area in which regions of high concentration level of glucose and regions of low concentration level of water alternately appear.

More preferably, it is possible to measure the photo-absorption distribution of hemoglobin at the same time in addition to the above-described apparatus system. It is known that the total amount of hemoglobin is increased in cancer tissue since the amount of blood flow is increased as neovasculars are proliferated in the tissue. Based on this phenomenon, it is possible to detect breast cancer at a higher accuracy by determining the presence or absence of an area where regions of high concentration level of hemoglobin and glucose and regions of low concentration level of water alternately appear.

The structure and operation principle of the tumor photo-detection device according to an embodiment will now be described.

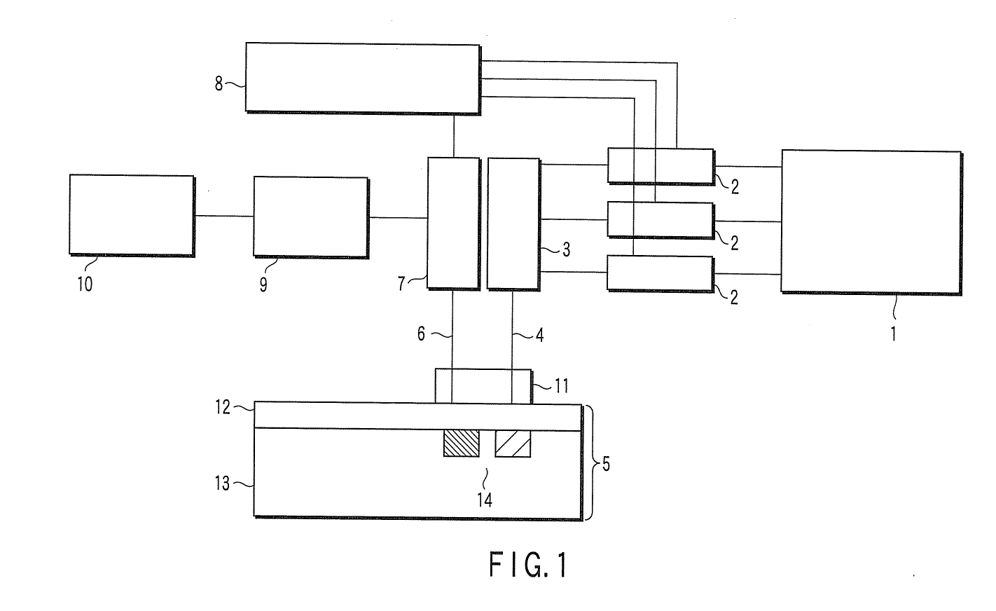

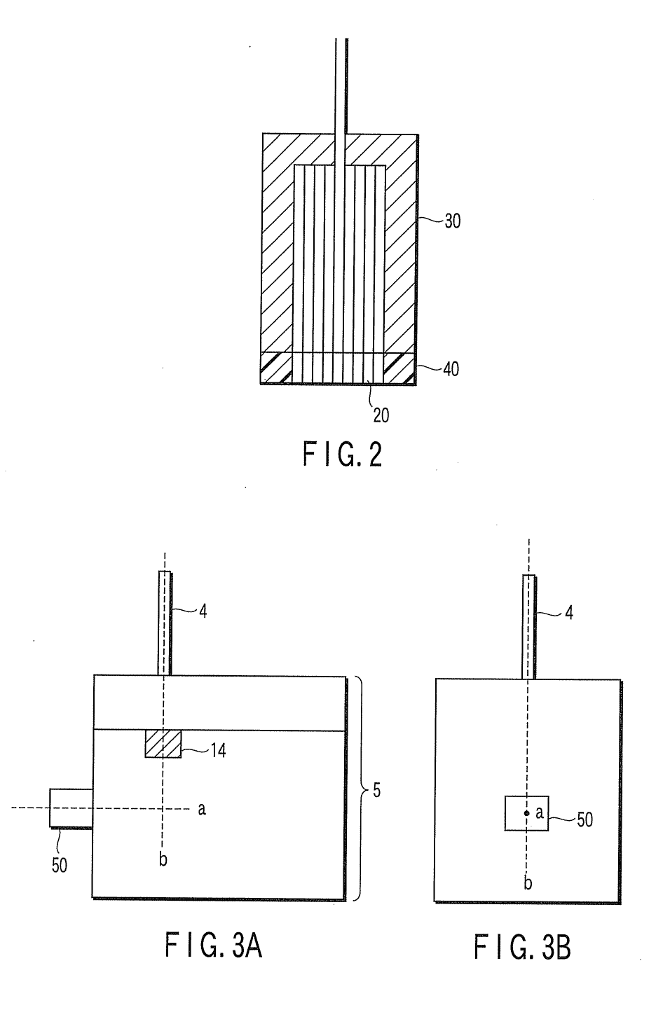

FIG. 1 shows the tumor photo-detection device according to the embodiment. Light of a wavelength within the photo-absorption band of a desired substance to be measured is radiated from each of 3 types of semiconductor lasers (LD) 2 connected to a function generator 1. As will now be described, in Example 1, hemoglobin, oxidized hemoglobin and glucose are selected as the above-mentioned substances to be measured, and a light beam of a wavelength within the photo-absorption band of each respective substance is irradiated from each respective one of the three types of LDs. The number of LDs can be increased or decreased as needed in accordance with the number of substances to be measured. The light beams irradiated from the LDs are overlaid with a light synthesizer-coupler 3 and the synthesized light is irradiated on organism tissue (living model sample 5 in Example 1) via a light-irradiating optical fiber 4 (an irradiator). It should be noted that a living model sample presented in Example 1 has a plurality of grooves 14 formed therein, where model blood is contained, and a thin plate 12, which indicates the same optical constant as that of lipid, is tightly set still on the sample. Next, the reflected light obtained from the organism tissue (that is, in Example 1, the living model sample 5) is detected with an OE converter 7 via one light-detecting optical fiber 6 (a detector). An OE output is connected to a lock-in amplifier 8, and a signal to an LD driver 2 is applied to an external trigger. In this manner, outputs from the LDs can be detected independently. Further, the OE output is set so as to switch from the lock-in amplifier 8 to an AC coupled amplifier 9, and the intensity of the signal is amplified with a main amplifier. After that, the signal is AD-converted with an AD converter 10 and then the converted signal is fed in a PC. The light-irradiating optical fiber 4 and light-detecting optical fiber 6 are fixed to a single probe 11 with a certain interval each other. FIG. 2 shows a cross section of the probe 11. As shown in this figure, a light-irradiating and light-detecting optical fiber 20 is placed inside of a metal cylinder 30, and an end of the probe is covered with a polymer cap 40.

Next, the increase and/or decrease of each output beam which were detected with the OE converter 7, each beam with a wavelength of the photo-absorption band of each substance to be measured, are analyzed (an analyzing unit). Subsequently, with the above-mentioned an irradiator, a detector and an analyzing unit, the organism tissue is analyzed in a number of regions. That is, in a number of regions of the organism tissue, the increase and/or decrease of each output beam which were detected with the OE converter 7, each beam with a wavelength of the photo-absorption band of each substance to be measured, are analyzed. Then, the distribution of the concentration level of the substances to be measured in each of the regions is operated (an operation unit). Lastly, the distribution of concentration level is analyzed to determine the presence or absence of an area where the plurality of substances are alternately observed (a determination unit).

On the other hand, the light energy accumulated on the compound by the photo-absorption generates acoustic wave, and the generated acoustic wave can be measured with an acoustic element. In order to detect the concentration level of a compound by an optoacoustic method, the light irradiating optical fiber and the acoustic element are arranged as shown in FIG. 3A and 3B. The light irradiating optical fiber 4 is placed above a groove 14 in which model blood of the living model sample 5 is included, and an acoustic element 50 is tightly attached to a surface vertical to the light incident surface via an acoustic matching layer. FIG. 3B is a front view of the acoustic element 50 shown in FIG. 3A.

In case of detecting and measuring acoustic wave, first, a plurality of lights are irradiated on organism tissue wherein the lights have wavelengths within the photo-absorption spectral regions of a plurality of desired substances to be measured (glucose, water and hemoglobin) and the intensity of the lights changes along with time (an irradiator). Second, an acoustic wave generated from the organism tissue is detected (a detector). Third, the site where the acoustic wave is generated is operated by measuring the arrival time of the ultrasonic wave detected by the acoustic element (an operation unit). Finally, the presence or absence of a tumor in the site is determined by constructing a spatial distribution image from the generation site and the amplitude value of the acoustic wave, and comparing between the spatial distribution value in a section to be examined in the spatial distribution image and the spatial distribution value in a normal part in the spatial distribution image (a determination unit).

It should be noted here that FIGS. 1 to 3 shows the structure of a tumor photo-detection device, which is merely an embodiment of the present invention. The tumor photo-detection device of the present invention covers all enabling embodiments that satisfy the structure of what is claimed.

### EXAMPLES

### Example 1

Two types of hemoglobin (oxidized and normal) and glucose were selected as the absorbers to be measured. In place of the organism tissue, the following model sample was prepared. That is, first, a silicone resin was selected as a base material. Then, a scattering material (10% lipid sphere dispersion liquid, product name: Intralipid) and an absorbing material (Near-infrared region dye, product name: Greenish Green) were dispersed in the silicone resin, and the resultant was cured, thus preparing simulated lipid. The simulated lipid exhibited the same optical constant (scattering coefficient and absorbing coefficient) as those of natural lipid. The simulated lipid is sliced into pieces of various thicknesses of 5 mm to 30 mm (in steps of 5 mm), and thus thin plates of the simulated lipid were manufactured. Subsequently, a silicone resin was selected as a base material as described above, and a block body which could contain a test sample was manufactured. In the surface portion of the block body, pits with various widths and lengths from 5 mm to 25 mm (in steps of 5 mm) and a constant depth of 5 mm were cut. FIG. 5 shows a top view of the sample model.

As the model bloods, two types of aqueous solutions were prepared. For the solutions, dyes exhibiting spectra that match the absorption spectra of the near-infrared regions (near 800 nm) of the hemoglobin and oxidized hemoglobin were selected, respectively. (Model 1: the aqueous solution of the hemoglobin, and Model 2: the aqueous solution of the oxidized hemoglobin.) Various amounts of glucose, that is, 0 mg/dl to 500 mg/dl (in steps of 100 mg/dl) were added to each of the model bloods, and thus model tumor components were prepared. Each model tumor component was injected to the pits formed in the block body carefully not to mix the air therein (Model 1 to the odd-numbered pits and Model 2 to the even-numbered pits). Then, the thin plates were placed on the block body and tightly attached thereto so that no air layer is created.

The wavelengths to be measured were set to 760 nm, 840 nm and 905 nm to match the absorption bands of the hemoglobin, oxidized hemoglobin and glucose, respectively. As the light sources, 3 near-infrared LDs (continuously oscillating LDs with their intensities modulated with sine waves with frequencies of 500 kHz, 600 kHz and 700 kHz, respectively) were selected. The modulated light beams output from the LDs were synthesized on a filter and the synthesized light was irradiated on the sample via a single optical fiber (quartz single core type having a diameter of 250 μm). Meanwhile, the output light from the sample was transferred with an optical fiber (plastic multicore type having a diameter of 500 μm), and it was detected with a system (of the OE detector that can detect an output of subnanoW to 10 mW) in which a logarithmic amplifier is connected to a high-speed response avalanche Si photodiode. The distance between the two optical fibers was set to 3 cm. The OE output was connected to the lock-in amplifier, and the output to an LD driver was applied to an external trigger, thus making it possible to detect the outputs of the LDs independently. The output from the lock-in amplifier is further amplified with the main amplifier, and then the amplified signal is AD-converted and fed in a PC.

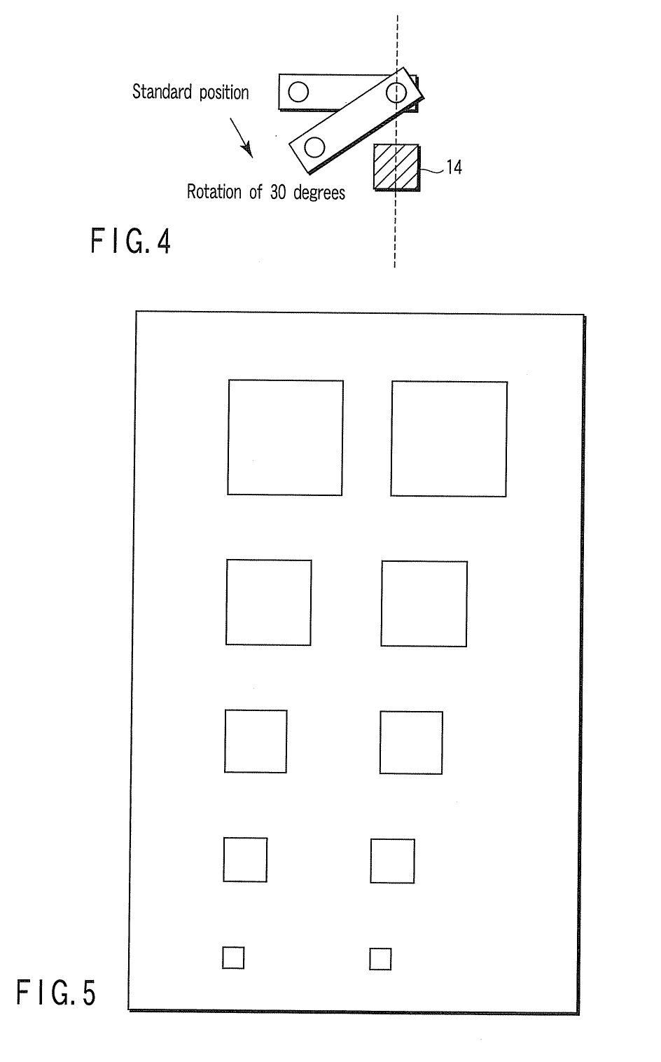

First, the optical output from each LD was detected with the lock-in amplifier while the probe was aligned on the position where there was no pit, and the intensity of the light passed through the model while there is no absorber was measured. The current values supplied to the respective LDs were adjusted so that the 3 LD outputs were approximately equalized (10 nanoW to submicroW level). The probe was rotated around the position of the optical source fiber. An example is shown in FIG. 4. This figure shows a model in which the position of the light source was not changed but only the position of the detector was changed, and this arrangement was carried out in order to maintain the light incident conditions as constant as possible. The probe was rotated in steps of 15 degrees. The probe started from the position where there is no pit (0 degree), passed through the position where there are pits and lastly moved to the position where there is no pit (180 degrees). The average of the measured values at 0 degree and 180 degrees was taken as a measurement result of the case where there is no absorber (reference value). The difference between the reference value and the measurement result at each position was taken as the change due to the photo-absorption. 6 types of thin plates having various thicknesses (from 5 mm to 30 mm, in steps of 5 mm) were prepared. In each of the thin plates, 5 types of pits having various widths (from 5 mm to 25 mm, in steps of 5 mm) were made. Then, the above-described 2 types of model tumor components (one only with the hemoglobin and the other only with the oxidized hemoglobin) having a glucose concentration level of 0 mg/dl were injected to these pits, respectively, which were then subjected to the measurements. For all of the 6 types of thin plates, the intensity of the signal for the 760-nm LD was reduced in the odd-numbered pits and that of the 840-nm LD was reduced in the even-numbered pits, but no change in intensity was observed in the other two LDs for the rest of the wavelengths. From the measurement results thus obtained, it was interpreted that the presence of the applicable absorbers was detected. As the width of pit becomes smaller from 25 mm to 5 mm, the level of the reduced signal intensity was lower. However, the level of the reduced signal intensity could be distinguished from the noise. Further, as the thin plate became thicker, the intensity of the light passed a position where there was no pit became significantly low. Therefore, the current supplied to the LD was increased to enhance the optical output.

Next, for the thin plates with a thickness of 15 mm and a pit width of 15 mm, model tumor components with various glucose concentration levels from 0 mg/dl to 500 mg/dl (in steps of 100 mg/dl) were injected to the respective pits, and then subjected to the measurements. Focusing on each LD intensity, it was observed that the signal intensity for the 760-nm and 905-nm LDs was reduced in the odd-numbered pits, whereas that of the 840-nm and 905-nm LDs was reduced in the even-numbered pits, but no substantial change was observed in the signal intensity for the other LD of the different wavelength. As the glucose concentration level was higher, the signal intensity for the 905-nm LD became smaller as compared to the reference value (in other words, glucose absorbed the 905-nm light). The signal intensity for each of the 760-nm and 840-nm LDs was not changed even if the glucose concentration level was changed. In reverse, the signal intensity for the 905-nm LD was not changed even if the concentration level of the oxidized hemoglobin or hemoglobin was changed.

From the results obtained above, it was found that even in the case where the oxidized hemoglobin or hemoglobin was co-present with glucose, the level of the photo-absorption due to glucose could be measured with the light with a wavelength of 905 nm.

Further, the relationship between the width of a pit of the block body and the signal intensity was examined. It was found that when the width of a pit was reduced, the signal intensity for all the types of LDs of these wavelengths was lowered, and therefore it was necessary to increase the original intensity of the respective LDs. On the other hand, when the thin plate was thicker, the signal intensity for all the types of LDs of these wavelengths was lowered as in the case where the width of a pit was reduced.

### Example 2

The device was evaluated using the same model simple as that of EXAMPLE 1.

The wavelengths to be measured were set to 760 nm, 840 nm and 1640 nm to match the absorption bands of the hemoglobin, oxidized hemoglobin and glucose, respectively. As the light sources, 3 near-infrared LDs (pulse-oscillating LDs at time intervals of 10 ns and a repeating frequency of 10 kHz) were selected. The modulated light beams output from the LDs were synthesized on a filter and the synthesized light was irradiated on the sample via a single optical fiber (quartz single core type having a diameter of 250 μm). Meanwhile, an acoustic element was tightly attached to a surface vertical to the light incident surface via an acoustic matching layer, the detection of acoustic wave was carried out. The acoustic signal was fed to the lock-in amplifier where the signal of the LD driver was applied as an external trigger, thus making it possible to detect the outputs of the LDs independently. The output from the lock-in amplifier is further amplified with the main amplifier, and then the amplified signal is AD-converted and taken in a PC. It should be noted that the probe used in EXAMPLE 1 was employed here as well in order to monitor the intensity of the light input to the sample for each LD separately, and the intensity of the incident light was monitored with high-speed response Si photodiodes (of 760 nm and 840 nm) or an InGaAs photodiode (of 1640 nm).

First, the light radiating fiber was placed on the position where there was no pit and then the optical output from each LD was detected with the lock-in amplifier. In this manner, the intensity of the light passed through the model while there was no absorber and the optoacoustic signal generated from the model were measured. The current values supplied to the respective LDs were adjusted while monitoring the light intensity so that the 3 LD outputs were approximately equalized (10 nanoW to submicrow level). The optoacoustic signal measured in this state was not synchronized with any of the 3 types of LDs, and therefore it was interpreted as background noise that is not originated from a particular absorber.

After that, the light radiating fiber was placed on the position where there were pits and the acoustic element was tightly attached at a relative position similar to that shown in FIG. 3A and 3B. Then, the optoacoustic signal was measured. 6 types of thin plates having various thicknesses (from 5 mm to 30 mm, in steps of 5 mm) were prepared. In each of the thin plates, 5 types of pits having various widths (from 5 mm to 25 mm, in steps of 5 mm) were made. Then, the above-described 2 types of model tumor components (one only with the hemoglobin and the other only with the oxidized hemoglobin) having a glucose concentration level of 0 mg/dl were injected to these pits, respectively, which were then subjected to the measurements. For all of the 6 types of thin plates, the intensity of the signal synchronizing with the frequency of the 760-nm LD was observed in the odd-numbered pits and that of the 840-nm LD was observed in the even-numbered pits. From the measurement results thus obtained, it was interpreted that the presence of the applicable absorbers was detected. As the width of pit becomes smaller from 25 mm to 5 mm, the level of the synchronizing acoustic signal was lower. However, it could be observed as it was distinguished from the background noise. Further, as the thin plate became thicker, the intensity of the light having passed a position where there was no pit became significantly low. Therefore, it was necessary to increase the current supplied to the LD to enhance the optical output.

Next, for the thin plates with a thickness of 15 mm and a pit width of 15 mm, model tumor components with various glucose concentration levels from 0 mg/dl to 500 mg/dl (in steps of 100 mg/dl) were injected to the respective pits, and then subjected to the measurements. Focusing on the acoustic signal synchronizing with each respective LD frequency, the intensity of the signal synchronizing with the frequencies of the 760-nm and 1640-nm LDs was observed in the odd-numbered pits, whereas that of the 840-nm and 1640-nm LDs was observed in the even-numbered pits. As the glucose concentration level was increased, the intensity of the signal synchronizing with the 1640-nm LD became stronger, but an SN ratio sufficient to determine the function type was not obtained. The intensity of the signal synchronizing with the 760-nm or 840-nm LD was not changed even if the glucose concentration level was changed. In reverse, the intensity of the signal synchronizing with the 1640-nm LD was not changed even if the concentration level of the oxidized hemoglobin or hemoglobin was changed.

From the results obtained above, it was found that even in the case where the oxidized hemoglobin or hemoglobin was co-present with glucose, the level of the photo-absorption due to glucose could be measured with the light with a wavelength of 1640 nm.

Further, the relationship between the width of a pit of the block body and the signal intensity was examined. It was found that when the width of a pit was reduced, the intensity of the signal synchronizing with all the types of LDs of these wavelengths was lowered, and therefore it was necessary to increase the original intensity of the respective LDs. On the other hand, when the thin plate was made thicker, the intensity of the signal synchronizing with all the types of LDs of these wavelengths was lowered as in the case where the width of a pit was reduced.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A tumor photo-detection device comprising:
i) an irradiator which irradiates first light with a wavelength within a photo-absorbing band of glucose and second light with a wavelength within a photo-absorbing band of water, on a plurality of regions of organism tissue;
ii) a detector which detects reflected light of the first light from the organism tissue and reflected light of the second light from the organism tissue;
iii) an operation unit configured to operate distributions of concentration levels of glucose and water in the plurality of regions by comparing the reflected light of the first light with that of the second light with regard to an increase or decrease in intensity; and
iv) a determination unit configured to determine presence or absence of an area where regions of high concentration level of glucose and regions of low concentration level of water appear alternately by analyzing the distributions of the concentration levels.

2. The device according to claim 1, wherein the irradiator overlays the first light and the second light on a same optical path.

3. The device according to claim 1, wherein the irradiator is fixed and the detector is rotated around the irradiator.

4. A tumor photo-detection device comprising:
i) an irradiator which irradiates first light with a wavelength within a photo-absorbing band of glucose, second light with a wavelength within a photo-absorbing band of water and third light with a wavelength within a photo-absorbing band of hemoglobin, on a plurality of regions of organism tissue;
ii) a detector which detects reflected light of the first light from the organism tissue, reflected light of the second light from the organism tissue and reflected light of the third light from the organism tissue;
iii) an operation unit configured to operate distributions of concentration levels of glucose, water and hemoglobin in the plurality of regions by comparing the reflected light of the first light, that of the second light and that of the third light with each other with regard to an increase or decrease in intensity; and
iv) a determination unit configured to determine presence or absence of an area where regions of high concentration levels of glucose and hemoglobin and regions of a low concentration level of water appear alternately by analyzing the distributions of the concentration levels.

5. The device according to claim 4, wherein the irradiator overlays the first light, second light and third light on a same optical path.

6. The device according to claim 4, wherein the irradiator is fixed and the detector is rotated around the irradiator.

7. An acoustic wave detection device comprising:
i) an irradiator which irradiates first light with a wavelength within a photo-absorbing band of glucose and second light with a wavelength within a photo-absorbing band of water, on a site to be measured in organism tissue;
ii) a detector which detects acoustic wave generated from the site to be measured;
iii) an operation unit configured to operate distributions of concentration levels of glucose and water in the site where the acoustic wave is generated by measuring an arrival time of an ultrasonic wave detected by the detector; and
iv) a determination unit configured to determine presence or absence of a tumor in the site by constructing a spatial distribution image from the generation site and the amplitude value of the acoustic wave, and comparing between the spatial distribution value in a section to be examined in the spatial distribution image and the spatial distribution value in a normal part in the spatial distribution image.
